(19) United States
(12) Patent Application Publication
(10) Pub. No.: US 2004/0236364 A1
(43) Pub. Date: Nov. 25, 2004

(54) Method of treating aneurysms with bioabsorbable polymers

(76) Inventor: Donald K. Jones, Lauderhill, FL (US)

Correspondence Address: Philip S. Johnson, Esq., Johnson & Johnson, One Johnson & Johnson Plaza, New Brunswick, NJ, 08933-7003, US

(21) Appl. No.: 10/443,582

(22) Filed: May 22, 2003

(51) Int. Cl. A61M 29/00

(52) U.S. Cl. 606/191

(57) ABSTRACT

A method of treating an aneurysm including the steps of introducing a delivery system into the body of a patient, delivering into the aneurysm an embolic device comprised of a bioabsorbable polymer for the purpose of promoting an inflammatory response, withdrawing the delivery system from the body of the patient, and instructing or advising the patient to avoid the use of an anti-inflammatory medication or agent in order to maintain the inflammatory response promoted by the polymeric embolic device.

### BACKGROUND OF THE INVENTION

1. Field of the Invention

The present invention concerns a novel method of treating an aneurysm, such as an aneurysm in a blood vessel of the brain, using an embolic device, and more particularly, an embolic device formed from a bioabsorbable polymer.

2. Description of the Prior Art

The use of vaso-occlusive medical devices and materials has become a standard for the treatment of vascular defects such as aneurysms. Injectable fluids, polymeric foams and beads, polymeric resins such as cyanoacrylate resins, mechanical vaso-occlusive embolic devices such as helically wound coils, ribbons, and braids, are only a few examples of the devices and materials used to treat aneurysms. Various shaped embolic coils are disclosed in the prior art, such as in U.S. Pat. No. 5,624,461, entitled “Three Dimensional In-filling Vaso-occlusive Coils,” which discloses a complex helically wound coil comprised of a primary helically wound coil which is then wound into a specific secondary shape, and U.S. Pat. No. 5,639,277, entitled “Embolic Coils With Offset Helical and Twisted Helical Shapes,” which discloses a helically wound coil in which the helix is wound in such a way as to have multiple axially offset, longitudinal or focal axes. The purpose of these medical devices and materials is to fill an aneurysm, reduce the flow of blood within the aneurysm, and ultimately cause the formation of a thrombus. The thrombus within the aneurysm further reduces the flow of blood into the aneurysm, thus reducing the pressure on the aneurysm wall and thereby reducing the likelihood the aneurysm will rupture.

Within the past decade, embolic devices have been constructed using absorbable bioactive, or bioabsorbable, polymers. When placed within an aneurysm, such polymeric embolic devices promote an inflammatory response. The bioabsorbable polymer of the embolic device is absorbed and is generally replaced with natural tissue by the natural wound-healing process as it is absorbed. The natural tissue, in combination with thrombus, is formed to thereby fill and eventually close the aneurysm. Recently developed bioabsorbable embolic coils are formed of a combination of bioabsorbable polymer and metallic filler material, with up to seventy percent of the coil being formed of bioabsorbable polymer. U.S. Pat. No. 6,423,085, entitled “Biodegradable Polymer Coils For Intraluminal Implants,” discloses a coil comprised in part of a bioabsorbable polymer.

Various methods have been used to deliver embolic devices to an aneurysm. One such method is disclosed in U.S. patent application Ser. No. 09/955,396, entitled “Method for Placing a Medical Agent into a Vessel of the Body,” filed on Sep. 18, 2001, and assigned to the same assignee as the present patent application and hereby incorporated by reference. The delivery system disclosed in U.S. Pat. No. 6,361,547, entitled “Embolic Coil Hydraulic Deployment System,” also assigned to the same assignee as the present patent application and hereby incorporated by reference, may be used as a delivery system to perform the method of delivery as set forth in the above-mentioned patent application. More particularly, the above-mentioned method of delivery and delivery system may be used to treat an aneurysm within the brain of a patient.

Optimally, the delivery to an aneurysm of an embolic device formed of a bioabsorbable polymer promotes the re-endothelialization of the aneurysm and accelerates wound healing of the aneurysm. If, however, an anti-inflammatory medication is used after the delivery of the polymeric embolic device, such medication may retard the localized inflammatory response and the bioabsorbable polymer of the embolic device may be absorbed prior to being replaced with natural tissue, resulting in an aneurysm which is incompletely filled. With an incompletely filled aneurysm, blood flow resumes within the aneurysm and largely defeats the intended purpose of the embolic device.

### SUMMARY OF THE INVENTION

In accordance with the present invention, there is provided a method of treating an aneurysm. The method includes the steps of introducing an embolic device delivery system into a blood vessel of a patient, advancing the delivery system along the blood vessel until the distal end of the delivery system is aligned with an aneurysm, delivering an embolic device comprised of a bioabsorbable polymer through the delivery system into the aneurysm, withdrawing the delivery system from the blood vessel of the patient, and cautioning the patient to limit the use of an anti-inflammatory medication, or an anti-inflammatory agent, for a predetermined period of time after the embolic device is delivered into the aneurysm.

In accordance with another aspect of the present invention, the bioabsorbable polymer is preferably comprised of polyglycolic acid, polylactic acid, or polyhydroxybutyrate/hydroxyvalerate copolymer. Alternatively, the bioabsorbable polymer may be comprised of poly-gycolic/poly-L-lactic acid copolymers, polycaprolactive, poly-L-lactide, polydioxanone, polycarbonates, polyanhydrides, or any combination thereof.

In accordance with a further aspect of the present invention, the anti-inflammatory medication or agent is preferably ibuprofen, aspirin, or caffeine. Alternatively, the anti-inflammatory medication or agent may also be diclofenac, diflunisal, etodolac, fenoprofen, flurbiprofen, indomethacin, ketoprofen, meclofenamate, nabumetone, naproxen, oxaprozin, phenylbutazone, piroxicam, sulindac, tenoxicam, tiaprofenic acid, tolmetin, aspirin and caffeine, aspirin and buffered caffeine, buffered aspirin, choline and magnesium salicylates, choline salicylate, magnesium salicylate, salsalate, sodium salicylate, or any combination thereof.

In accordance with yet another aspect of the present invention, there is provided a method of treating an aneurysm including the steps of introducing a vaso-occlusive device delivery system into a vessel of a body of a patient, advancing the delivery system along the vessel until the distal end of the delivery system is aligned with an aneurysm, delivering a vaso-occlusive device, such as an embolic coil comprised of a bioabsorbable polymer, through the delivery system into the aneurysm, withdrawing the delivery system from the vessel of the body, and instructing the patient to avoid the use of an anti-inflammatory medication for a predetermined period of time after the vaso-occlusive device is delivered into the aneurysm.

In accordance with another aspect of the present invention, there is provided a method of treating an aneurysm including the steps of introducing a medical device delivery system into a blood vessel, advancing the delivery system along the blood vessel until the distal end of the delivery system reaches an aneurysm, delivering a medical device comprised of a bioabsorbable polymer through the delivery system into the aneurysm, withdrawing the delivery system from the blood vessel, and advising to avoid the use of an anti-inflammatory agent.

In accordance with still another aspect of the present invention, there is provided a method of treating an aneurysm comprising the steps of introducing an inflammatory response-inducing material into an aneurysm and instructing the patient to restrict the use of an anti-inflammatory medication.

In accordance with yet another aspect of the present invention, there is provided a method of embolizing a blood vessel including the steps of introducing a vaso-occlusive device delivery system into a blood vessel of a body of a patient, advancing the delivery system along the blood vessel until the distal end of the delivery system reaches a site to be embolized, delivering a vaso-occlusive device, such as an embolic coil comprised of a bioabsorbable polymer, through the delivery system into the blood vessel, withdrawing the delivery system from the blood vessel of the body, and instructing the patient to limit the use of an anti-inflammatory medication for a predetermined period of time after the vaso-occlusive device is delivered into the blood vessel.

### BRIEF DESCRIPTION OF THE DRAWINGS

FIG. 1 is a partial sectional view showing a hydraulic embolic coil delivery system used to deliver a bioabsorbable embolic coil to an aneurysm;

FIG. 2 is an enlarged, partial sectional view of a positioning catheter, used in conjunction with the embolic coil delivery system of FIG. 1, having a normally deflected distal section and a connector;

FIG. 3 is an enlarged, partial sectional view of the positioning catheter in use to deliver a bioabsorbable embolic coil into an aneurysm;

FIGS. 4-8 are diagrammatic sequential views of a method of placing bioabsorbable embolic coils within an aneurysm; and,

FIG. 9 is a flow chart illustrating the method steps of a preferred embodiment of the present invention.

### DESCRIPTION OF THE PREFERRED EMBODIMENT

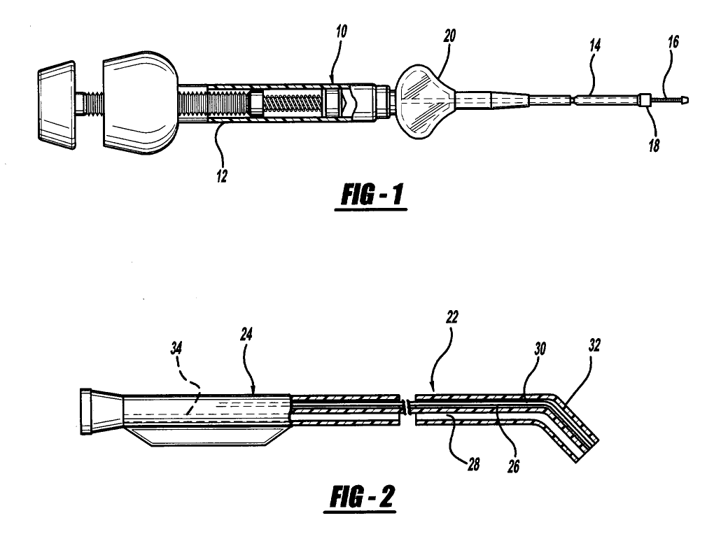

FIG. 1 illustrates a hydraulic embolic coil delivery system 10 which includes a syringe 12 coupled to the proximal end of a delivery catheter 14. An embolic coil 16, formed of a bioabsorbable polymer, is positioned and tightly held within the lumen of the distal section 18 of the delivery catheter 14 until the delivery catheter is activated for release of the bioabsorbable embolic coil. As shown, the delivery system 10 includes a winged hub 20, which aids in the introduction of the delivery catheter 14 into a blood vessel of a patient, and more particularly a blood vessel within the brain of the patient. A more detailed description of the delivery system 10 is disclosed in U.S. Pat. No. 6,113,622, entitled “Embolic Coil Hydraulic Deployment System,” assigned to the same assignee as the present patent application and hereby incorporated by reference.

FIG. 2 illustrates a positioning catheter 22 having its proximal end connected to the distal end of a connector 24. The positioning catheter 22 includes a first lumen 26 and a second lumen 28. The first lumen 26 extends from the proximal end to the distal end of the positioning catheter 22. A pre-shaped retaining wire 30 extends through the first lumen 26 and is bent away from the longitudinal axis of the positioning catheter 22 causing the distal section 32 of the positioning catheter to be deflected angularly away from the centerline of the positioning catheter. The second lumen 28 extends the entire length of the positioning catheter 22 and communicates with a lumen 34, which extends from the proximal end to the distal end of the connector 24. A more detailed description of the positioning catheter 22 is disclosed in the aforementioned U.S. patent application Ser. No. 09/955,396.

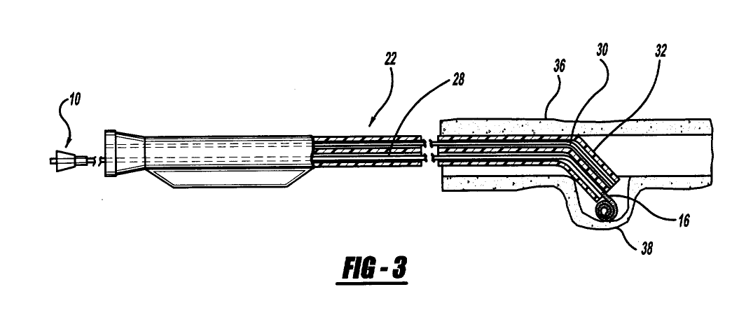

FIG. 3 illustrates the positioning catheter 22 inserted into a blood vessel 36 of the brain of a patient. The pre-shaped retaining wire 30 deflects the distal section 32 of the positioning catheter 22 so as to align the positioning catheter with the mouth of the aneurysm 38. The delivery system 10 is shown inserted through the second lumen 28 of the positioning catheter 22 in order to deliver the bioabsorbable embolic coil 16 into the aneurysm 38.

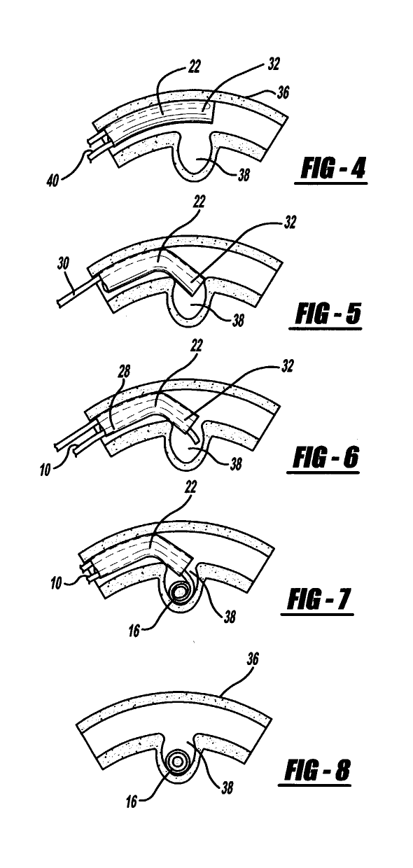

FIGS. 4 through 8 illustrate the operation of the positioning catheter 22, used in conjunction with the delivery system 10, for the delivery of the bioabsorbable embolic coil 16 into the aneurysm 38. As illustrated in FIG. 4, the positioning catheter 22 is inserted into a blood vessel 36 over a straightening wire 40. The positioning catheter 22 is then positioned such that the distal section 32 of the positioning catheter is proximate the aneurysm 38. Next, the straightening wire 40 is removed and, as illustrated in FIG. 5, the pre-shaped retaining wire 30 deflects the distal section 32 of the positioning catheter 22 and thereby aligns the distal section of the positioning catheter with the aneurysm 38. Once the distal section 32 of the positioning catheter 22 has been properly positioned, the delivery system 10 may then be inserted into the second lumen 28 and through the distal section of the positioning catheter, as illustrated in FIG. 6. Then, the bioabsorbable embolic coil 16 may be placed into the aneurysm 38 and released from the delivery system 10, as illustrated in FIG. 7. The delivery system 10 may then be removed, and this process may be repeated until a sufficient number of bioabsorbable embolic coils 16 have been placed into the aneurysm 38. When the aneurysm 38 has been sufficiently filled with bioabsorbable embolic coils, the delivery system 10 is withdrawn from the positioning catheter 22. Thereafter, the straightening wire 40 is again inserted into the second lumen 28 thereby causing the distal section 32 of the positioning catheter 22 to straighten within the blood vessel 36. As illustrated in FIG. 8, the positioning catheter 22 may then be withdrawn from the blood vessel 36, leaving the bioabsorbable embolic coils 16 within the aneurysm 38.

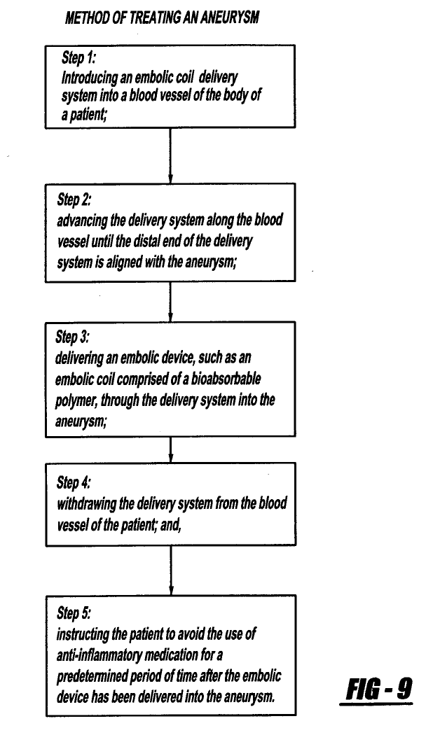

FIG. 9 illustrates the method steps of the preferred embodiment of the present invention. The steps include the following:

1. introducing an embolic coil delivery system, as previously described, into a blood vessel of the body of a patient;

2. advancing the delivery system along the blood vessel until the distal end of the delivery system is aligned with the aneurysm;

3. delivering an embolic device, such as an embolic coil comprised of a bioabsorbable polymer, through the delivery system into the aneurysm;

4. withdrawing the delivery system from the blood vessel of the patient; and,

5. instructing the patient to avoid the use of anti-inflammatory medication for a predetermined period of time after the embolic device has been delivered into the aneurysm.

The embolic coil delivery system serves to deliver a bioabsorbable embolic coil into an aneurysm, however, any medical or vaso-occlusive device comprised of, at least in part, a bioabsorbable polymer, or any other inflammatory response-inducing material, may be delivered into the aneurysm. The bioabsorbable polymer is preferably comprised of polyglycolic acid, polylactic acid, or polyhydroxybutyrate/hydroxyvalerate copolymers, however, the bioabsorbable polymer may also be comprised of poly-gycolic/poly-L-lactic acid copolymers, polycaprolactive, poly-L-lactide, polydioxanone, polycarbonates, polyanhydrides, or any combinations thereof. The bioabsorbable polymer may also include proteins such as collagen, fibrinogen, fibronectin, vitronectin, laminin, or any combination thereof.

Alternatively, the positioning catheter and delivery system described above may be used to embolize a blood vessel by introducing a vaso-occlusive device, such as an embolic coil comprised of a bioabsorbable polymer, into the blood vessel. The patient is then instructed to limit the use of anti-inflammatory medication until the blood vessel has been sufficiently embolized.

The patient may be cautioned to restrict or limit the use of anti-inflammatory medication for a predetermined period of time. Preferably, the patient is instructed to entirely avoid the use of any anti-inflammatory medication, such as ibuprofen, aspirin, or caffeine, for such period of time. Other anti-inflammatory medications or agents which should be avoided include diflunisal, etodolac, fenoprofen, flurbiprofen, indomethacin, ketoprofen, meclofenamate, nabumetone, naproxen, oxaprozin, phenylbutazone, piroxicam, sulindac, tenoxicam, tiaprofenic acid, tolmetin, aspirin and caffeine, aspirin and buffered caffeine, buffered aspirin, choline and magnesium salicylates, choline salicylate, magnesium salicylate, salsalate, sodium salicylate, or any combination thereof.

As indicated, the patient is instructed to avoid the use of anti-inflammatory medication for a predetermined period of time. The predetermined period of time is dependant upon the absorption rate of the bioabsorbable polymer of the embolic device. The bioabsorbable polymer of the embolic device will generally absorb during the first six months after delivery of the embolic device into an aneurysm, and may be absorbed within one or two days after delivery into the aneurysm. Therefore, in the case of an embolic device formed from a bioabsorbable polymer which would be absorbed within a period of thirty days, a physician would instruct the patient to avoid the use of anti-inflammatory medication during such thirty-day period. This period of time provides the time necessary for the embolic device to produce its intended results, i.e. promote inflammation with resultant tissue in-growth.

By avoiding the use of anti-inflammatory medication, the embolic device formed with a bioabsorbable polymer promotes a localized inflammatory response prior to the absorption of the polymer. The inflammatory response then triggers the natural wound healing process, causing tissue in-growth within the aneurysm. While the bioabsorbable polymer of the embolic device is being absorbed, natural tissue replaces the volume of space vacated by the bioabsorbable polymer to thereby aid in the complete healing of the aneurysm.

Although a preferred embodiment of the invention has been described, it is to be understood that various modifications may be made by those skilled in the art without departing from the scope of the following claims.

That which is claimed is:

1. A method of treating an aneurysm comprising the steps of:
introducing an embolic device delivery system having a distal end into a blood vessel of a patient;
advancing the delivery system along the blood vessel until the distal end of the delivery system is aligned with an aneurysm;
delivering an embolic device comprised of a bioabsorbable polymer through the delivery system into the aneurysm;
withdrawing the delivery system from the blood vessel of the patient; and,
cautioning the patient to limit the use of an anti-inflammatory medication for a predetermined period of time after the embolic device is delivered into the aneurysm.

2. A method of treating an aneurysm as defined in claim 1, wherein the bioabsorbable polymer is comprised of polyglycolic acid.

3. A method of treating an aneurysm as defined in claim 1, wherein the bioabsorbable polymer is comprised of polylactic acid.

4. A method of treating an aneurysm as defined in claim 1, wherein the bioabsorbable polymer is comprised of polyhydroxybutyrate/hydroxyvalerate copolymer.

5. A method of treating an aneurysm as defined in claim 1, wherein the bioabsorbable polymer is selected from a group consisting of polyglycolic acid, polylactic acid, poly-gycolic/poly-L-lactic acid copolymers, polycaprolactive, polyhydroxybutyrate/hydroxyvalerate copolymers, poly-L-lactide, polydioxanone, polycarbonates, polyanhydrides, and any combination thereof.

6. A method of treating an aneurysm as defined in claim 1, wherein the anti-inflammatory medication is ibuprofen.

7. A method of treating an aneurysm as defined in claim 1, wherein the anti-inflammatory medication is Aspirin.

8. A method of treating an aneurysm as defined in claim 1, wherein the anti-inflammatory medication is Caffeine.

9. A method of treating an aneurysm as defined in claim 1, wherein the anti-inflammatory medication is selected from a group consisting of Caffeine, diclofenac, diflunisal, etodolac, fenoprofen, flurbiprofen, ibuprofen, indomethacin, ketoprofen, meclofenamate, nabumetone, naproxen, oxaprozin, phenylbutazone, piroxicam, sulindac, tenoxicam, tiaprofenic acid, tolmetin, Aspirin, Aspirin and Caffeine, Aspirin and buffered Caffeine, buffered Aspirin, Choline and magnesium salicylates, choline salicylate, magnesium salicylate, salsalate, sodium salicylate, and any combination thereof.

10. A method of treating an aneurysm comprising the steps of:
introducing a vaso-occlusive device delivery system having a distal end into a vessel of a body of a patient;
advancing the delivery system along the vessel until the distal end of the delivery system is aligned with an aneurysm;
delivering a vaso-occlusive device through the delivery system into the aneurysm;
withdrawing the delivery system from the vessel of the body; and,
instructing the patient to avoid the use of an anti-inflammatory medication for a predetermined period of time after the vaso-occlusive device is delivered into the aneurysm.

11. A method of treating an aneurysm as defined in claim 10, wherein the vaso-occlusive device is comprised of a bioabsorbable polymer.

12. A method of treating an aneurysm as defined in claim 11, wherein the vaso-occlusive device takes the form of an embolic coil.

13. A method of treating an aneurysm as defined in claim 12, wherein the bioabsorbable polymer is comprised of polyglycolic acid.

14. A method of treating an aneurysm as defined in claim 12, wherein the bioabsorbable polymer is comprised of polylactic acid.

15. A method of treating an aneurysm as defined in claim 12, wherein the bioabsorbable polymer is comprised of polyhydroxybutyrate/hydroxyvalerate copolymer.

16. A method of treating an aneurysm as defined in claim 12, wherein the bioabsorbable polymer is selected from a group consisting of polyglycolic acid, polylactic acid, poly-gycolic/poly-L-lactic acid copolymers, polycaprolactive, polyhydroxybutyrate/hydroxyvalerate copolymers, poly-L-lactide, polydioxanone, polycarbonates, polyanhydrides, and any combination thereof.

17. A method of treating an aneurysm as defined in claim 12, wherein the anti-inflammatory medication is ibuprofen.

18. A method of treating an aneurysm as defined in claim 12, wherein the anti-inflammatory medication is Aspirin.

19. A method of treating an aneurysm as defined in claim 12, wherein the anti-inflammatory medication is Caffeine.

20. A method of treating an aneurysm as defined in claim 12, wherein the anti-inflammatory medication is selected from a group consisting of Caffeine, diclofenac, diflunisal, etodolac, fenoprofen, flurbiprofen, ibuprofen, indomethacin, ketoprofen, meclofenamate, nabumetone, naproxen, oxaprozin, phenylbutazone, piroxicam, sulindac, tenoxicam, tiaprofenic acid, tolmetin, Aspirin, Aspirin and Caffeine, Aspirin and buffered Caffeine, buffered Aspirin, Choline and magnesium salicylates, choline salicylate, magnesium salicylate, salsalate, sodium salicylate, and any combination thereof.

21. A method of treating an aneurysm comprising the steps of:
introducing a medical device delivery system having a distal end into a blood vessel;
advancing the delivery system along the blood vessel until the distal end of the delivery system reaches an aneurysm;
delivering a medical device comprised of a bioabsorbable polymer through the delivery system into the aneurysm;
withdrawing the delivery system from the blood vessel; and,
advising to avoid the use of an anti-inflammatory agent.

22. A method of treating an aneurysm as defined in claim 21, wherein the bioabsorbable polymer is comprised of polyglycolic acid.

23. A method of treating an aneurysm as defined in claim 21, wherein the bioabsorbable polymer is comprised of polylactic acid.

24. A method of treating an aneurysm as defined in claim 21, wherein the bioabsorbable polymer is comprised of polyhydroxybutyrate/hydroxyvalerate copolymer.

25. A method of treating an aneurysm as defined in claim 21, wherein the anti-inflammatory agent is ibuprofen.

26. A method of treating an aneurysm as defined in claim 21, wherein the anti-inflammatory agent is Aspirin.

27. A method of treating an aneurysm as defined in claim 21, wherein the anti-inflammatory agent is Caffeine.

28. A method of treating an aneurysm comprising the steps of:
introducing an embolic coil delivery system having a distal end into a vessel of a body of a patient;
advancing the delivery system along the vessel until the distal end of the delivery system is aligned with an aneurysm;
delivering an embolic coil comprised of polyglycolic acid through the delivery system into the aneurysm;
withdrawing the delivery system from the vessel of the body; and,
instructing the patient to avoid the use of ibuprofen.

29. A method of treating an aneurysm comprising the steps of:
introducing an inflammatory response-inducing material into an aneurysm; and,
instructing a patient to restrict the use of an anti-inflammatory medication.

30. A method of treating an aneurysm as defined in claim 29, wherein the inflammatory response-inducing material is a medical device.

31. A method of treating an aneurysm as defined in claim 30, wherein the medical device is an embolic device taking the form of an embolic coil.

32. A method of treating an aneurysm as defined in claim 31, wherein the embolic device is comprised of a bioabsorbable polymer.

33. A method of treating an aneurysm as defined in claim 32, wherein the bioabsorbable polymer is comprised of polyglycolic acid.

34. A method of treating an aneurysm as defined in claim 32, wherein the bioabsorbable polymer is comprised of polylactic acid.

35. A method of treating an aneurysm as defined in claim 32, wherein the bioabsorbable polymer is comprised of polyhydroxybutyrate/hydroxyvalerate copolymer.

36. A method of treating an aneurysm as defined in claim 32, wherein the anti-inflammatory medication is ibuprofen.

37. A method of treating an aneurysm as defined in claim 32, wherein the anti-inflammatory medication is Aspirin.

38. A method of treating an aneurysm as defined in claim 32, wherein the anti-inflammatory medication is Caffeine.

39. A method of embolizing a blood vessel comprising the steps of:
introducing a vaso-occlusive device delivery system having a distal end into a blood vessel of a body of a patient;
advancing the delivery system along the blood vessel until the distal end of the delivery system reaches a site to be embolized;
delivering a vaso-occlusive device through the delivery system into the blood vessel;
withdrawing the delivery system from the blood vessel of the body; and,
instructing the patient to limit the use of an anti-inflammatory medication for a predetermined period of time after the vaso-occlusive device is delivered into the blood vessel.

40. A method of embolizing a blood vessel as defined in claim 39, wherein the vaso-occlusive device is comprised of a bioabsorbable polymer.

41. A method of embolizing a blood vessel as defined in claim 40, wherein the vaso-occlusive device takes the form of an embolic coil.

42. A method of embolizing a blood vessel as defined in claim 41, wherein the bioabsorbable polymer is comprised of polyglycolic acid.

43. A method of embolizing a blood vessel as defined in claim 41, wherein the bioabsorbable polymer is comprised of polylactic acid.

44. A method of embolizing a blood vessel as defined in claim 41, wherein the bioabsorbable polymer is comprised of polyhydroxybutyrate/hydroxyvalerate copolymer.

45. A method of embolizing a blood vessel as defined in claim 41, wherein the bioabsorbable polymer is selected from a group consisting of polyglycolic acid, polylactic acid, poly-gycolic/poly-L-lactic acid copolymers, polycaprolactive, polyhydroxybutyrate/hydroxyvalerate copolymers, poly-L-lactide, polydioxanone, polycarbonates, polyanhydrides, and any combination thereof.

46. A method of embolizing a blood vessel as defined in claim 41, wherein the anti-inflammatory medication is ibuprofen.

47. A method of embolizing a blood vessel as defined in claim 41, wherein the anti-inflammatory medication is Aspirin.
